(19) United States
(12) Patent Application Publication
(10) Pub. No.: US 2024/0065297 A1
(43) Pub. Date: Feb. 29, 2024

(54) FERMENTED BEVERAGE COMPOSITION

(71) Applicant: SOCIETE DES PRODUITS NESTLE S.A., Vevey (CH)

(72) Inventors: JIYUAN ZHANG, Lausanne (CH); CYRIL MOCCAND, Lully (CH); CATHERINE NGOM-BRU, Mezieres (CH); AXEL ANDRE, Chexbres (CH)

(21) Appl. No.: 18/002,750

(22) PCT Filed: Jun. 22, 2021
(86) PCT No.: PCT/EP2021/066903

§ 371 Date: Dec. 21, 2022

(30) Foreign Application Priority Data
Jun. 24, 2020 (EP) 20181980.2

(51) Int. Cl. A23L 2/38; A23L 2/385; A23L 2/52

(52) U.S. Cl. CPC A23L 2/382; A23L 2/385; A23L 2/52

(57) ABSTRACT

The present invention relates to a beverage composition comprising fermented cascara or fermented cascara extract. A further aspect of the invention is a method of preparing a beverage composition. A still further aspect of the invention is the yeast Pichia kluyveri NYSC 5485 (CNCM I-5525).

### CROSS REFERENCE TO RELATED APPLICATIONS

The present application is a National Stage of International Application No. PCT/EP2021/066903, filed on Jun. 22, 2021, which claims priority to European Patent Application No. 20181980.2, fled on Jun. 24, 2020, the entire contents of which are being incorporated herein by reference.

### SEQUENCE LISTING

The instant application contains a Sequence Listing which has been submitted electronically in ASCII format and is hereby incorporated by reference in its entirety. Said ASCII copy, created on Jul. 18, 2023, is named Sequence_Listing.txt and is 29,837.071 bytes in size.

### FIELD OF THE INVENTION

The present invention relates to a beverage composition comprising fermented cascara or fermented cascara extract. A further aspect of the invention is a method of preparing a beverage composition.

### BACKGROUND OF THE INVENTION

Fruitiness in low-alcohol beverages is sought after by consumers, but many people would prefer not to consume beverages with added flavouring, especially synthetic flavourings. The fruity perception in most low-alcohol beverages is often achieved by adding high-sugar containing fruit concentrates, resulting in higher sugar content which is not always desirable.

Cascara beverages are increasing popular. Cascara, which is the Spanish word for husk, is the outer part of the coffee cherry. The production of cascara beverages utilises this part of the coffee cherry that is often discarded. Typically the outer part of the coffee cherry is removed from the coffee bean and dried in the sun before being used to brew beverages in a similar fashion to tea. Cascara beverages have a subtle and often tart sweetness rather than bitter taste profile making it ideal for refreshing beverages. However, some consumers perceive a “stewed tea” flavour in cascara that they do not enjoy.

Any reference to prior art documents in this specification is not to be considered an admission that such prior art is widely known or forms part of the common general knowledge in the field. As used in this specification, the words “comprises”, “comprising”, and similar words, are not to be interpreted in an exclusive or exhaustive sense. In other words, they are intended to mean “including, but not limited to”.

### SUMMARY OF THE INVENTION

An object of the present invention is to improve the state of the art and to provide a new product to overcome at least some of the inconveniences described above, or at least to provide a useful alternative. The object of the present invention is achieved by the subject matter of the independent claims. The dependent claims further develop the idea of the present invention.

Accordingly, the present invention provides in a first aspect a beverage composition comprising fermented cascara or fermented cascara extract wherein the weight ratio of 2-phenylethyl acetate to benzaldehyde is greater than 1.

A second aspect of the invention provides a container for use in a beverage preparation device, the container containing the beverage composition of the invention.

A third aspect of the invention provides a starter culture comprising Pichia kluyveri NYSC 5485 (CNCM 1-5525).

In a fourth aspect, the invention provides a method of preparing a beverage composition comprising;
a. steeping cascara in water to form an aqueous cascara extract and steeped cascara; and
b. fermenting the aqueous cascara extract in the presence or absence of the steeped cascara;
wherein the fermentation is performed by yeast in the absence of acetic acid bacteria.

Surprisingly, the inventors found that by fermenting cascara with yeast they are able to provide an enhancement in the sensorial perception. In particular, the perception of floral and fruity notes were significantly improved, corresponding to increasing concentrations of floral and fruity-related volatiles. The levels of individual aroma compounds such as acetate esters gave a well-balanced aroma profile without an acetone-like off odor or vinegar/beer-like/winy fermented notes. By fermenting in the absence of acetic acid bacteria they avoided generating the acid “bite” present in traditional fermented drinks such as kombucha.

### BRIEF DESCRIPTION OF THE DRAWINGS

FIG. 1 shows sensory profiles of infusions of cascara from Brazil fermented with different yeasts. Left-hand column; Acid, Bitter. Sweet. Right-hand column; Fruity, Fermented, Floral. The samples are compared to the reference set as 0 and are “Control 24H”, treated as per the fermentation samples but with no yeast; “NYSC5485”, fermentation with Pichia kluyveri NYSC 5485; “NCYC246”, fermentation with P. kluyveri NCYC 246; and “S33”, fermentation with Saccharomyces cerevisiae S-33.

FIG. 2 shows sensory profiles of infusions of cascara from Mexico fermented with different yeasts. Left-hand column; Acid, Bitter, Sweet. Right-hand column; Fruity, Fermented, Floral. Fermentation samples labeled as for FIG. 1.

FIG. 3 shows (from top to bottom) the levels of 2-phenylethyl acetate, isopentyl acetate and isobutyl acetate in parts per million of infusions of cascara from Mexico after fermentation with different yeasts. Fermentation samples labeled as for FIG. 1.

### DETAILED DESCRIPTION OF THE INVENTION

Consequently the present invention relates in part to a beverage composition comprising (for example consisting of) fermented cascara or fermented cascara extract wherein the weight ratio of 2-phenylethyl acetate to benzaldehyde is greater than 1. For example the weight ratio of 2-phenylethyl acetate to benzaldehyde may be greater than 1.5, 3, 6, 10, 15, 20, 25, 30, 40 or 50. For example the weight ratio of 2-phenylethyl acetate to benzaldehyde may be from 1 to 500, for example from 5 to 400, for example from 10 to 200, for example from 20 to 100, for example from 20 to 50, for further example from 22 to 40. In an aspect of the invention, the weight ratio of 2-phenylethyl acetate to benzaldehyde is greater than 0.8, for example greater than 0.9. In an aspect of the invention the weight ratio of 2-phenylethyl acetate to benzaldehyde is from 0.8 to 500, for example from 0.9 to 100.

2-phenylethyl acetate has an attractive, floral, rose-like aroma. Increased concentrations of 2-phenylethyl acetate correspond to enhanced floral notes. Benzaldehyde provides a useful denominator for a ratio with 2-phenylethyl acetate as it is naturally present in cascara but is not greatly influenced by fermentation.

In the context of the present invention, cascara is the outer part of the coffee cherry. It is distinct from cascara sagrada, the bark of Rhamnus purshiana. The coffee cherry is the fruit of the Coffea plant, a member of the Rubiaceae family. Coffee cherries are sometimes referred to as coffee berries. The coffee cherries may be the fruit of Arabica (Coffea arabica). Robusta (Coffea canephora) or mixtures of these.

Two types of cascara are available depending on the processing method. Husk cascara is produced by drying whole coffee cherries and then dehulling them. For example the husk cascara according to the invention may be produced from sun dried coffee cherries. The husk cascara comprises the outer skin, pulp, mucilage and parchment of the coffee cherry. Pulp cascara is produced by washing coffee cherries and de-pulping them, usually in the wash water. The separated pulp is then dried. For example the pulp cascara according to the invention may be sun dried. Pulp cascara comprises the outer skin and the pulp of the coffee cherry. The cascara according to the invention may be selected from the group consisting of husk cascara, pulp cascara and combinations of these. The cascara according to the invention may be pulp cascara.

To optimize the flavour of the cascara it should ideally be produced from ripe coffee cherries. It is difficult to avoid some unripe coffee cherries being included in the harvest but, for example, the cascara according to the invention may be produced from predominantly ripe coffee cherries. For example, at least 80% of the coffee cherries used to produce the cascara may be red in colour, for example at least 90%.

In the context of the present invention the term “fermentation” refers to a process in which the activity of microorganisms brings about a change (typically a desirable change) to a foodstuff or beverage. The fermentation may be with yeasts and/or bacteria. The fermentation may be anaerobic or aerobic. Fermentation is one of the oldest means of preserving and enhancing foods.

The term “fermented cascara” refers to cascara which has been subjected to a process in which the activity of microorganisms such as yeasts brings about a chemical change in the organic components of the cascara, typically a desirable change. The fermented cascara may have been fermented in an aqueous medium, for example with yeast. The fermented cascara may be a dried cascara which had been added to an aqueous fermentation medium and fermented before being separated from the aqueous fermentation medium and dried. The fermented cascara may have been fermented in a “solid-state” fermentation, for example adding a starter culture of yeast to coffee cherry pulp.

The term “cascara extract” refers to material extracted from cascara, for example an aqueous extract of cascara. The term “fermented cascara extract” refers to an extract of cascara where the cascara extract has been fermented, for example an aqueous cascara extract where the aqueous extract has been fermented, such as by yeast. The fermented cascara extract may be a dried fermented cascara extract.

Benzaldehyde, a commonly found organic compound in natural sources, with a characteristic almond-like odour and medium odour threshold. Naturally present in cascara, green and roasted coffee beans, benzaldehyde is not considered as key coffee odorant due to its relatively low concentration.

Isopentyl acetate, also known as isoamyl acetate, naturally occurs in bananas. At appropriate concentrations is provides a pleasant fruity note, resembling bananas and pears. In an embodiment the weight ratio of isopentyl acetate to benzaldehyde is greater than 1. For example the weight ratio of isopentyl acetate to benzaldehyde may be greater than 1.5, 3, 6, 10, 15, 20, 25, 30, 40, 50, 60 or 70. In an embodiment, the weight ratio of isopentyl acetate to benzaldehyde may be from 1 to 500, for example from 5 to 400, for example from 10 to 200, for example from 20 to 100, for example from 50 to 300, for further example from 40 to 200.

Isobutyl acetate provides a pleasant fruity note. In an embodiment the weight ratio of isobutyl acetate to benzaldehyde is greater than 0.5. For example the weight ratio of isobutyl acetate to benzaldehyde may be greater than 1, 2, 3 or 4. The weight ratio of isobutyl acetate to benzaldehyde may be from 0.5 to 20, for example from 1 to 15, for example from 2 to 10 for further example from 3 to 6. 3-Methylbutanol is a common volatile produced through fermentation, and provides mainly pungent and alcoholic notes. Therefore, maximizing the ratio of isopentyl acetate to 3-methlybutanol would result in a beverage with higher level of fruitiness with minimal fermented notes. In an embodiment the weight ratio of isopentyl acetate to 3-methylbutanol is greater than 0.5. For example the weight ratio of isopentyl acetate to 3-methylbutanol may be greater than 1, 2, 3 or 4. The weight ratio of isopentyl acetate to 3-methylbutanol may be from 0.5 to 40, for example from 1 to 30, for example from 2 to 20, for example from 3 to 15, for further example from 3 to 4.

Both isopentyl acetate and hexyl acetate provide fruity notes, although isopentyl acetate has much higher odour strength than hexyl acetate. Therefore, a high ratio of isopentyl acetate to hexyl acetate indicates the intensity of fruitiness in the beverages. In an embodiment the weight ratio of isopentyl acetate to hexyl acetate is greater than 10. For example the weight ratio of isopentyl acetate to hexyl acetate may be greater than 100 or greater than 500. The weight ratio of isopentyl acetate to hexyl acetate may be from 100 to 1000. The weight ratio of isopentyl acetate to hexyl acetate may be from 200 to 3000, for example from 250 to 2000, for further example from 500 to 1500. In an embodiment, the level of hexyl acetate in the beverage composition is less than 0.1 ppm.

In an embodiment the weight ratio of 2-phenylethanol to benzaldehyde is greater than 2.7 (for example greater than 2.8, 2.9, 3.0). In an embodiment the weight ratio of ethanol to benzaldehyde is less than 50000 (for example less than 40000, for further example less than 30000). In an embodiment the weight ratio of 2-phenylethanol to benzaldehyde is greater than 2.7 (for example greater than 2.8, 2.9, 3.0) and the weight ratio of ethanol to benzaldehyde is less than 50000 (for example less than 40000, for further example less than 30000).

In an embodiment the weight ratio of ethyl acetate to benzaldehyde is greater than 50. For example the weight ratio of ethyl acetate to benzaldehyde may be greater than 100 or greater than 500. The weight ratio of ethyl acetate to benzaldehyde may be from 50 to 2000. The weight ratio of ethyl acetate to benzaldehyde may be from 300 to 1500, for example from 500 to 1100. An appropriate ratio of ethyl acetate to benzaldehyde is associated with a fruity aroma. Too high levels of ethyl acetate may result in an unpleasant ethereal aroma. In an embodiment, the beverage composition is selected from the group consisting of a ready to drink beverage, a beverage concentrate, a soluble beverage powder, dried aromatic plant material and combinations of these. In an embodiment, the beverage composition is a beverage extractable composition for the preparation of a beverage. Beverage extractable compositions for the preparation of a beverage include compositions which dissolve completely such as soluble beverage powders as well as compositions which combine extractable and insoluble components such as dried cascara. The beverage composition may comprise added vitamins. The beverage composition may comprise added antioxidants. The beverage composition may comprise added flavours. However, fermentation according to the invention generates attractive fruity aromas and so added flavours may not be required. In an embodiment the beverage composition does not comprise added flavours. The beverage composition may consist of coffee bean and cascara components. The beverage composition may consist of cascara components. The beverage composition may be free from coffee bean components, for example free from roast and ground coffee beans or free from extracts of roast and ground coffee beans.

In an embodiment the beverage composition is a mixture of dried fermented cascara extract and roast and ground coffee, for example to be brewed with water to form a beverage. In an embodiment the beverage composition is a mixture of dried fermented cascara extract, dried fermented cascara and roast and ground coffee, for example to be brewed with water to form a beverage.

The ready to drink beverage may be a fermented cascara infusion or a water flavoured with fermented cascara. The ready to drink beverage may additionally comprise other components such as flavourings or stabilizers. The ready to drink beverage may be carbonated. The ready to drink beverage may additionally comprise coffee (for example cold-brew coffee), tea (for example an aqueous extract of the leaves, leaf-buds, twigs or stems of the plant Camellia sinensis.

The dried aromatic plant material may be selected from the group consisting of cascara, roast and ground coffee, dried tea (e.g. dried leaves, leaf-buds, twigs or stems of the plant Camellia sinensis, dried herbal tea (e.g. dried flowers, fruit, leaves, seeds or roots of plants other than Camellia sinensis) and combinations of these. In an embodiment the beverage composition is a mixture of dried fermented cascara and roast and ground coffee, for example to be brewed with water to form a beverage.

Beverage preparation devices (for example beverage preparation machines) which accommodate extractable portioned ingredients provide a convenient method of preparing beverages. Such portioned ingredients are generally packed in a container, configured for example as a pod, pad, sachet, pouch, capsule or the like. An aspect of the invention provides a container for use in a beverage preparation device, the container containing the beverage composition of the invention. The container being for the preparation of a beverage when inserted into a beverage preparation device. The container may for example be a beverage capsule, among other configurations. In an embodiment, the container contains the beverage composition of the invention. For example the container may contain dried fermented cascara; dried fermented cascara extract; a mixture of dried fermented cascara and roast and ground coffee; a mixture of dried fermented cascara extract and roast and ground coffee; or a mixture of dried fermented cascara extract, dried fermented cascara and roast and ground coffee.

In an embodiment, the beverage composition has an ethanol content below 1.2 wt %, for example below 0.5 wt %, for example below 0.2 wt %, for further example below 0.05 wt %.

An aspect of the invention provides a method of preparing a beverage composition comprising;
a. steeping cascara in water to form an aqueous cascara extract and steeped cascara; and
b. fermenting the aqueous cascara extract in the presence or absence of the steeped cascara;
wherein the fermentation is performed by yeast in the absence of acetic acid bacteria.

The beverage composition prepared by the method of the invention may be a cascara beverage, for example a beverage comprising fermented cascara or fermented cascara extract. The beverage composition prepared by the method of the invention may have an ethanol content below 1.2 wt %, for example below 0.5 wt %, for example below 0.2 wt %, for further example below 0.05 wt %. In an embodiment, the beverage composition prepared by the method of the invention is the beverage composition of the invention.

The term steeping refers to immersing and soaking a material in liquid. The cascara may be steeped in water for at least 5 minutes, for example at least 10 minutes, for example at least 30 minutes, for example at least 60 minutes, for further example at least 120 minutes. During the steeping process, components of the cascara are extracted into the water. The steeping may be performed at a temperature between 4° C. and 98° C., for example between 20° C. and 95° C., for example between 60° C. and 95° C. Although cascara contains fermentable sugar, further fermentable sugar may be added to the aqueous cascara extract, for example sucrose or glucose. The fermentable sugar may be fructose or glucose. For example fermentable sugar may be added to the aqueous cascara extract at a level of between 2 and 10 wt. %, for example between 3 and 7 wt. %. The cascara may be ground before steeping.

When fermentation is to be performed in the absence of the steeped cascara, the cascara may for example be steeped in water using a continuous counter-current extraction system. A further option is to steep the cascara in water using a batch process, with the steeped cascara being separated from the aqueous cascara extract using a filter system. The ratio of cascara on a dry basis to water during steeping may be between 1:1 and 1:100 by weight, for example between 1:2 and 1:95 by weight, for example between 1:10 and 1:90 by weight, for example between 1:30 and 1:70 by weight, for further example between 1.3 and 1:6.

After steeping, the temperature of the aqueous cascara extract is adjusted to a temperature suitable for yeast growth, for example between 20° C. and 37° C., for example between 25° C. and 35° C., for example between 28° C. and 32° C. The temperature may for example be adjusted using a laminar flow heat exchanger. Yeast is added to the aqueous cascara extract to commence fermentation. Fermenting the aqueous cascara extract may be performed at a temperature between 25° C. and 35° C., for example between 28° C. and 32° C. Fermenting the aqueous cascara extract may be performed for at least 2 hours, for example at least 6 hours, for example at least 12 hours, for example at least 24 hours, for further example at least 48 hours.

The fermentation may be performed at a pH between 3 and 8, for example between 4 and 6.

The fermentation may be performed in the presence of a nitrogen source such as yeast extract or yeast peptone. The fermentation may be performed in the presence of added amino acids such as valine, leucine, isoleucine and/or phenylalanine, for example at a level of between 0.05 wt. % and 0.1 wt. % of the aqueous cascara extract.

The fermentation may be performed in anaerobic, microaerobic or aerobic conditions. For example, the partial oxygen pressure in the fermenter may be from 0 to 30%, for example from 1 to 20%, for further example from 2 to 10%.

Preferably the cascara has not been subjected to chemical processing such as hydrolysis before fermentation.

The fermentation may be performed by adding one or more yeasts at an initial combined yeast level of at least 10³ CFU/g, for example at least 10⁴ CFU/g, for example at least 10⁵ CFU/g, for example at least 10⁶ CFU/g, for further example at least 10⁷ CFU/g.

The fermentation is performed in the absence of acetic acid bacteria to avoid generating an acid “bite” in the flavour profile. In an embodiment the fermentation is performed in the absence of lactic acid bacteria.

In an embodiment the fermentation is performed in the absence of Saccharomyces subspecies. For example the fermentation may be performed in the absence of Saccharomyces cerevisiae. S. cerevisiae is used for winemaking and beer brewing. In addition to generating alcohol which may not be desired, S. cerevisiae generates a “fermented”, winey, yeasty flavour note rather than a fresh fruity note.

In an embodiment the fermentation is performed in the absence of a yeast selected from the group consisting of Zygosaccharomyces bailiff, Schizosaccharvmyces pombe, Torulaspora delbreuckii, Rhodotorula mucilaginosa, Brettanomyces bruxellensis and Candida stellate. These yeasts are commonly found in kombucha “tea-fungus” cultures.

Surprisingly, the inventors found that fermentation of cascara with Pichia kluyveri gave particularly good results. A beverage composition comprising cascara or cascara extract fermented with Pichia kluyveri had a pleasant fruity and floral flavour. Pichia kluyveri occurs naturally around the world, for example on olives, grapes and coffee. In an embodiment of the method of the invention, the yeast comprises Pichia kluyveri. For example, more than 50% of the yeast colony forming units present during fermentation may be Pichia kluyveri. For example, more than 50% of the micro-organism colony forming units present during fermentation may be Pichia kluyveri. For example, essentially all the yeast colony forming units present during fermentation may be Pichia kluyveri, For further example, essentially all the micro-organism colony forming units present during fermentation may be Pichia kluyveri.

Pichia kluyveri NCYC 246 (alternatively designated CBS 188) is the type-strain of Pichia kluyveri. It is publicly available, for example from the National Collection of Yeast Cultures, Quadram Institute Bioscience, Norwich, UK.

In an embodiment the yeast is Pichia kluyveri NCYC 246. For example more than 50% of the yeast colony forming units present during fermentation may be Pichia kluyveri NCYC 246. For example, more than 50% of the micro-organism colony forming units present during fermentation may be Pichia kluyveri NCYC 246. For example, essentially all the yeast colony forming units present during fermentation may be Pichia kluyveri NCYC 246. For further example, essentially all the micro-organism colony forming units present during fermentation may be Pichia kluyveri NCYC 246.

The inventors have found that especially good results were obtained by fermenting cascara with Pichia kluyveri NYSC 5485. Pichia kluyveri NYSC 5485 not only provides strong fruity and floral sensory characteristics but also reduces bitterness. In particular, the bitter “stewed-tea” note of cascara was reduced.

Pichia kluyveri is widely distributed in nature, for example being found on fruits such as coffee cherries and olives. Pichia kluyveri NYSC 5485, collected and isolated from a coffee fermentation in Nicaragua, was deposited with the Collection Nationale de Cultures de Microorganismes (CNCM), Institut Pasteur, 25 rue du Docteur Roux, F-75724 PARIS Cedex 15, France, on 23 Jun. 2020 and given the deposit number CNCM 1-5525.

In an embodiment, the yeast is Pichia kluyveri NYSC 5485 (CNCM 1-5525). For example more than 50% of the yeast colony forming units present during fermentation may be Pichia kluyveri NYSC 5485 (CNCM 1-5525). For example, more than 50% of the micro-organism colony forming units present during fermentation may be Pichia kluyveri NYSC 5485 (CNCM 1-5525). For example, essentially all the yeast colony forming units present during fermentation may be Pichia kluyveri NYSC 5485 (CNCM 1-5525), For further example, essentially all the micro-organism colony forming units present during fermentation may be Pichia kluyveri NYSC 5485 (CNCM 1-5525).

An aspect of the invention is Pichia kluyveri NYSC 5485 (CNCM 1-5525). An aspect of the invention is a yeast comprising a genetic sequence having at least 95% identity (for example at least 98% or 99% identity) to SEQ ID NO:1, a genetic sequence having at least 95% identity (for example at least 98% or 99% identity) to SEQ ID NO:2, a genetic sequence having at least 95% identity (for example at least 98% or 99% identity) to SEQ ID NO:3, a genetic sequence having at least 95% identity (for example at least 98% or 99% or 99.5% identity) to SEQ ID NO:4, a genetic sequence having at least 95% identity (for example at least 98% or 99% identity) to SEQ ID NO:5, a genetic sequence having at least 95% identity (for example at least 98% or 99% identity) to SEQ ID NO:6, a genetic sequence having at least 95% identity (for example at least 98% or 99% identity) to SEQ ID NO:7, a genetic sequence having at least 95% identity (for example at least 98% or 99% identity) to SEQ ID NO:8, a genetic sequence having at least 95% identity (for example at least 98% or 99% identity) to SEQ ID NO:9, a genetic sequence having at least 95% identity (for example at least 98% or 99% identity) to SEQ ID NO:10, a genetic sequence having at least 95% identity (for example at least 98% or 99% identity) to SEQ ID NO:11, and a genetic sequence having at least 95% identity (for example at least 98% or 99% identity) to SEQ ID NO:12.

An aspect of the invention provides a starter culture comprising Pichia kluyveri NYSC 5485 (CNCM 1-5525). The starter culture may comprise at least 10³ CFU/g of Pichia kluyveri NYSC 5485 (CNCM 1-5525), for example at least 10⁴ CFU/g of Pichia kluyveri NYSC 5485 (CNCM 1-5525), for example at least 10⁵ CFU/g of Pichia kluyveri NYSC 5485 (CNCM 1-5525), for example at least 10⁶ CFU/g of Pichia kluyveri NYSC 5485 (CNCM 1-5525), for example at least 10⁷ CFU/g of Pichia kluyveri NYSC 5485 (CNCM 1-5525), for example at least 10⁸ CFU/g of Pichia kluyveri NYSC 5485 (CNCM 1-5525), for further example at least 10⁹ CFU/g of Pichia kluyveri NYSC 5485 (CNCM 1-5525). The starter culture may be obtained by drying a concentrated yeast culture, for example spray drying the concentrated yeast culture together with maltodextrin. The starter culture may be stored frozen before being unfrozen and multiplied for use, for example the starter culture may be frozen at −80° C. in a vial with glycerol. The term “starter culture” refers to a composition comprising live microorganisms that are capable of initiating or effecting fermentation of organic material, optionally after being cultivated in a separate starter medium for obtaining a high-density culture. Accordingly, in an embodiment, a starter culture of the invention may be a high-density culture obtained by propagating a starter culture in a suitable medium. A starter culture according to the present invention may also contain, in addition to microorganisms, buffering agents and growth stimulating nutrients or preservatives or other carriers.

The genetic sequence of Pichia kluyveri NYSC 5485 (CNCM 1-5525) was analysed using PacBio sequencing technology. DNA purification was performed using a Qiagen Gentra Puregene Yeast kit. Twelve DNA fragments were identified. These may be individual chromosomes; 11 chromosomes and 1 mitochondrion DNA (SEQ ID NO:4). The genetic sequences are filed in electronic form as SEQ ID NO:1-SEQ ID NO:12. In an embodiment of the method of the invention, the yeast comprises a genetic sequence having at least 95% identity (for example at least 98% or 99% identity) to SEQ ID NO:1, SEQ ID NO:2, SEQ ID NO:3, SEQ ID NO:5, SEQ ID NO:6, SEQ ID NO:7, SEQ ID NO:8, SEQ ID NO:9, SEQ ID NO:10, SEQ ID NO:11 or SEQ ID NO:12. For example the yeast may comprise a genetic sequence having at least 95% identity (for example at least 98% or 99% identity) to a sequence selected from the group consisting of SEQ ID NO:1, SEQ ID NO:2, SEQ ID NO:3, SEQ ID NO:5, SEQ ID NO:6, SEQ ID NO:7, SEQ ID NO:8, SEQ ID NO:9, SEQ ID NO:10, SEQ ID NO:11 and SEQ ID NO:12.

In an embodiment of the method of the invention, the yeast comprises a genetic sequence having at least 95% identity (for example at least 98% or 99% identity) to SEQ ID NO:1, a genetic sequence having at least 95% identity (for example at least 98% or 99% identity) to SEQ ID NO:2, a genetic sequence having at least 95% identity (for example at least 98% or 99% identity) to SEQ ID NO:3, a genetic sequence having at least 95% identity (for example at least 98% or 99% identity) to SEQ ID NO:4, a genetic sequence having at least 95% identity (for example at least 98% or 99% identity) to SEQ ID NO:5, a genetic sequence having at least 95% identity (for example at least 98% or 99% identity) to SEQ ID NO:6, a genetic sequence having at least 95% identity (for example at least 98% or 99% identity) to SEQ ID NO:7, a genetic sequence having at least 95% identity (for example at least 98% or 99% identity) to SEQ ID NO:8, a genetic sequence having at least 95% identity (for example at least 98% or 99% identity) to SEQ ID NO:9, a genetic sequence having at least 95% identity (for example at least 98% or 99% identity) to SEQ ID NO:10, a genetic sequence having at least 95% identity (for example at least 98% or 99% identity) to SEQ ID NO:11 and a genetic sequence having at least 95% identity (for example at least 98% or 99% identity) to SEQ ID NO:12.

An aspect of the invention provides a method of preparing a beverage composition comprising; a) steeping cascara in water to form an aqueous cascara extract and steeped cascara; and b) fermenting the aqueous cascara extract in the presence or absence of the steeped cascara; wherein the fermentation is performed by a yeast comprising a genetic sequence having a global nucleotide identity at least 98% (for example at least 99%) to the combination of SEQ ID NOs:1-12. By the term “combination of SEQ ID NOs:1-12” it is meant that the sequence identity comparison is performed against all the individual DNA fragments as if concatenated into a single sequence.

An embodiment of the invention provides a method of preparing a beverage composition comprising; a) steeping cascara in water to form an aqueous cascara extract and steeped cascara; and b) fermenting the aqueous cascara extract in the presence or absence of the steeped cascara; wherein the fermentation is performed by yeast comprising a genetic sequence having at least 95% identity (for example at least 98% or 99% identity) to SEQ ID NO:1, a genetic sequence having at least 95% identity (for example at least 98% or 99% identity) to SEQ ID NO:2, a genetic sequence having at least 95% identity (for example at least 98% or 99% identity) to SEQ ID NO:3, a genetic sequence having at least 95% identity (for example at least 98% or 99% or 99.5% identity) to SEQ ID NO:4, a genetic sequence having at least 95% identity (for example at least 98% or 99% identity) to SEQ ID NO:5, a genetic sequence having at least 95% identity (for example at least 98% or 99% identity) to SEQ ID NO:6, a genetic sequence having at least 95% identity (for example at least 98% or 99% identity) to SEQ ID NO:7, a genetic sequence having at least 95% identity (for example at least 98% or 99% identity) to SEQ ID NO:8, a genetic sequence having at least 95% identity (for example at least 98% or 99% identity) to SEQ ID NO:9, a genetic sequence having at least 95% identity (for example at least 98% or 99% identity) to SEQ ID NO:10, a genetic sequence having at least 95% identity (for example at least 98% or 99% identity) to SEQ ID NO:11 and a genetic sequence having at least 95% identity (for example at least 98% or 99% identity) to SEQ ID NO:12.

In an embodiment, the yeast comprises a genetic sequence having a global nucleotide identity at least 98% (for example at least 99%) to the combination of SEQ ID NOs:1-12.

In an embodiment the yeast comprises a genetic sequence having at least 98% identity to SEQ ID NO:1, a genetic sequence having at least 98% identity to SEQ ID NO:2, a genetic sequence having at least 98% identity to SEQ ID NO:3, a genetic sequence having at least 99% identity to SEQ ID NO:5, a genetic sequence having at least 98% identity to SEQ ID NO:6, a genetic sequence having at least 99% identity to SEQ ID NO:7, a genetic sequence having at least 98% identity to SEQ ID NO:8, a genetic sequence having at least 98% identity to SEQ ID NO:9, a genetic sequence having at least 99% identity to SEQ ID NO:10, a genetic sequence having at least 98% identity to SEQ ID NO:11 and a genetic sequence having at least 98% identity to SEQ ID NO:12.

In an embodiment the yeast comprises a genetic sequence having at least 98% (for example at least 99%) identity to SEQ ID NO:1. In an embodiment the yeast comprises a genetic sequence having at least 98% (for example at least 99%) identity to SEQ ID NO:2. In an embodiment the yeast comprises a genetic sequence having at least 98% (for example at least 99%) identity to SEQ ID NO:3. In an embodiment the yeast comprises a genetic sequence having at least 99% identity to SEQ ID NO:5. In an embodiment the yeast comprises a genetic sequence having at least 98% (for example at least 99%) identity to SEQ ID NO:6. In an embodiment the yeast comprises a genetic sequence having at least 99% identity to SEQ ID NO:7. In an embodiment the yeast comprises a genetic sequence having at least 98% (for example at least 99%) identity to SEQ ID NO:8. In an embodiment the yeast comprises a genetic sequence having at least 98% (for example at least 99%) identity to SEQ ID NO:9. In an embodiment the yeast comprises a genetic sequence having at least 99% identity to SEQ ID NO:10. In an embodiment the yeast comprises a genetic sequence having at least 98% (for example at least 99%) identity to SEQ ID NO:11. In an embodiment the yeast comprises a genetic sequence having at least 98% (for example at least 99%) identity to SEQ ID NO:12.

In an embodiment, the aqueous cascara extract is separated from the steeped cascara after fermentation. For example the steeped cascara may be separated from the aqueous cascara extract using a filter system. After fermentation, the aqueous cascara extract becomes fermented cascara extract.

The fermented cascara extract may be packaged as a ready-to-drink beverage, for example packaged in a bottle or a can. Additional ingredients such as flavours and preservatives may be added to the fermented cascara extract before it is packaged as a ready-to-drink beverage. The fermented cascara extract may be heat-treated to deactivate or kill the yeast and any spoilage organisms before being packaged. The fermented cascara extract may be passed through a filter to remove the yeast and any spoilage organisms before being packaged.

In an embodiment the aqueous cascara extract is concentrated after being fermented. After fermentation the aqueous cascara extract may be concentrated to form a liquid concentrate, for example to be packaged and sold as such, or as an intermediate product for the manufacture of ready-to-drink products, for example in a different location. The aqueous cascara extract after fermentation (for example the fermented cascara extract) may be concentrated to form a powder, such as a soluble beverage powder. For example, the aqueous cascara extract after fermentation (the fermented cascara extract) may be dried by spray drying or freeze drying to form a soluble beverage powder. For example, the aqueous cascara extract after fermentation may be spray dried together with a carrier such as maltodextrin to form a soluble beverage powder. The soluble beverage powder may be packaged and sold as such, or it may be used as an intermediate product for the manufacture of ready-to-drink products, for example in a different location. The soluble beverage powder may be filled into a container for use in a beverage preparation device. The soluble beverage powder may be combined with other ingredients, for example dry ingredients such as tea, soluble coffee or roast and ground coffee. For example, the soluble beverage powder may be combined with roast and ground coffee and filled into a container for use in a beverage preparation device. The soluble beverage powder may be combined with milk powder or beverage creamer powder and optionally sugar to form a beverage mix. After fermenting the aqueous cascara extract in the presence of the steeped cascara, the steeped cascara becomes fermented cascara. In an embodiment, the steeped cascara, present during the fermentation of the aqueous cascara extract, is separated from the aqueous cascara extract after fermentation and then dried, for example using a rotary drier. The dried steeped cascara may be filled into a container for use in a beverage preparation device, or filled into a permeable bag such as a “tea-bag”. The dried steeped cascara may be combined with other dry ingredients such as tea, soluble coffee or roast and ground coffee.

In an embodiment, the steeped cascara, present during the fermentation of the aqueous cascara extract, is dried together with the aqueous cascara extract after fermentation, for example by freeze drying or spray drying. The dried steeped cascara (e.g. dried fermented cascara) together with dried aqueous cascara extract after fermentation (e.g. dried fermented cascara extract) may be filled into a container for use in a beverage preparation device, or filled into a permeable bag such as a “tea-bag”. The dried steeped cascara and dried aqueous cascara extract after fermentation may be combined with other dry ingredients such as tea, soluble coffee or roast and ground coffee.

The fermented cascara may be obtained in a “solid-state” fermentation, for example adding a starter culture of yeast to coffee cherry pulp. The term “solid-state” fermentation refers to fermentation taking place on solid materials. There is liquid water present in a solid-state fermentation, but the term is used in contrast to a liquid fermentation such as may occur in aqueous solution in a fermentation vessel.

An aspect of the invention provides a method of preparing a beverage composition comprising;
adding a starter culture of yeast to coffee cherry pulp, wherein the starter culture provides at least 10⁴ CFU (for example at least 10⁵ CFU, for further example at least 10⁶ CFU) of yeast per gram of coffee cherry pulp;
fermenting the coffee cherry pulp;
drying the fermented coffee cherry pulp to form fermented cascara; and either-extracting the fermented cascara with water to form a ready-to-drink beverage, or filling the fermented cascara into a container, the container being for the preparation of a beverage when inserted into a beverage preparation device.

The starter culture of yeast may be added as a liquid or a powder to the coffee cherry pulp. The coffee cherry pulp may have a moisture content of between 45 and 55 wt. % when the starter culture of yeast is added. The fermentation may occur at a solids content greater than 40 wt. %, for example greater than 45 wt. %, for further example greater than 50 wt. %.

Drying the fermented coffee cherry pulp may commence while some fermentation continues. For example, the coffee cherry pulp may be placed in a container such as a tank, the starter culture added and then fermentation allowed to proceed for 1-4 days before the fermented coffee cherry pulp is removed from the container and dried. For further example, the starter culture may be added to the coffee cherry pulp before the coffee cherry pulp is slowly dried, for example on a rack or suspended tray. Once fermentation has started, the coffee cherry pulp may be slowly dried whilst fermentation continues. For example the fermented coffee cherry pulp may be dried to a moisture content of below 20 wt. % (for example below 15 wt. %) over a period of between 1 and 30 days, for example between 4 and 20 days. The drying temperature may be less than about 40° C., for example between about 25° C. and 35° C. A fan can be used to blow dry air over the material until the coffee cherry pulp is dry. The fermented coffee cherry pulp may be dried in the sun.

The yeast may be Pichia kluyveri, for example Pichia kluyveri NYSC 5485.

The container for the preparation of a beverage when inserted into a beverage preparation device may be in the form of a pod, pad, sachet, pouch, capsule or the like.

The coffee cherry pulp according to the solid-state fermentation method may be essentially free from coffee beans, for example it may have been processed by a pulping machine which squeezes the cherries between fixed and moving surfaces and separates the coffee beans. The coffee cherry pulp may for example contain less than 10 wt. % coffee beans, for example less than 5 wt. % coffee beans, for example less than 1 wt. % coffee beans.

The coffee cherry pulp according to the solid-state fermentation method may be provided in the form of coffee cherries comprising coffee beans. The coffee beans may be separated from the coffee cherry pulp after fermentation.

The ready-to-drink beverage may be filled into a bottle or can. The fermented cascara may be combined with other extractable materials such as tea or roast and ground coffee before being extracted with water to form a ready-to-drink beverage.

The fermented cascara may be combined with roast and ground coffee in a container for the preparation of a beverage when inserted into a beverage preparation device.

Those skilled in the art will understand that they can freely combine all features of the present invention disclosed herein. In particular, features described for the product of the present invention may be combined with the method of the present invention and vice versa. Further, features described for different embodiments of the present invention may be combined. Where known equivalents exist to specific features, such equivalents are incorporated as if specifically referred to in this specification.

Further advantages and features of the present invention are apparent from the figures and non-limiting examples.

### EXAMPLES

### Example 1: Method of Fermenting Cascara

Two different cascara origins were used in the experiment; cascara harvested from wet processing in 2018 from Brazil and Mexico and then dried. A hot infusion was firstly made by adding 20 g cascara in 1000 ml of water at 90° C., and incubating for 1 h when the temperature drops below 30° C. The reference cascara infusion (Ref) was made by discarding the cascara and subjecting the infused water to filter sterilization (0.2 pm).

The fermented cascara was made by fermenting the infusion with different starter cultures, including Pichia kluyveri NYSC 5485 (CNCM 1-5525), P. kluyveri NCYC 246 (the type strain of Pichia kluyveri), commercial brewer's yeast Saccharomyces cerevisiae (SafAle S-33, LeSaffre, France) and a symbiotic culture of bacteria and yeasts (SCOBY) pellicle from Kombucha tea fermentation. All strains (except for SCOBY) were grown from glycerol vial in a food-grade yeast-peptone liquid medium, and inoculated in the reference cascara brew at log 6.5 (CFU/g). All the inoculated infusions were stored at microaerobic condition at 30° C. for 24 h. After the incubation, the cascara was discarded and infused water underwent filter sterilization (0.2 um). In addition, an un-fermented infusion was also done by incubating the hot infusion at 30° C. for 24 h without any inoculation as a negative control (Control 24H). All final products were subjects to filter sterilization for sensory evaluation. The viability of the different starter cultures was tested by fermenting in a yeast growth medium.

### Example 2: Sensory Analysis of Cascara Fermented with Different Yeasts

The sensory profiles of different variations of cascara infusions were evaluated by a panel of ten members. The evaluation was divided into two sessions based on the sources of cascara used, i.e., Brazil and Mexico cascara. In each session, a comparative profiling analysis was conducted between the reference infusion and other variations, including fermented variants of P. kluyveri NYSC 5485, P. kluyveri NCYC 246, S. cerevisiae S-33, SCOBY pellicle, as well as the non-fermented negative control. All samples were served at room temperature and seven sensory attributes were evaluated by the panel, including overall flavor intensity, fruity, floral, fermented, acid, sweet, and bitter notes. For each attribute, the difference with the reference was rated on a structured intensity scale going from −3 (much less) to 3 (much more), with the reference set as 0.

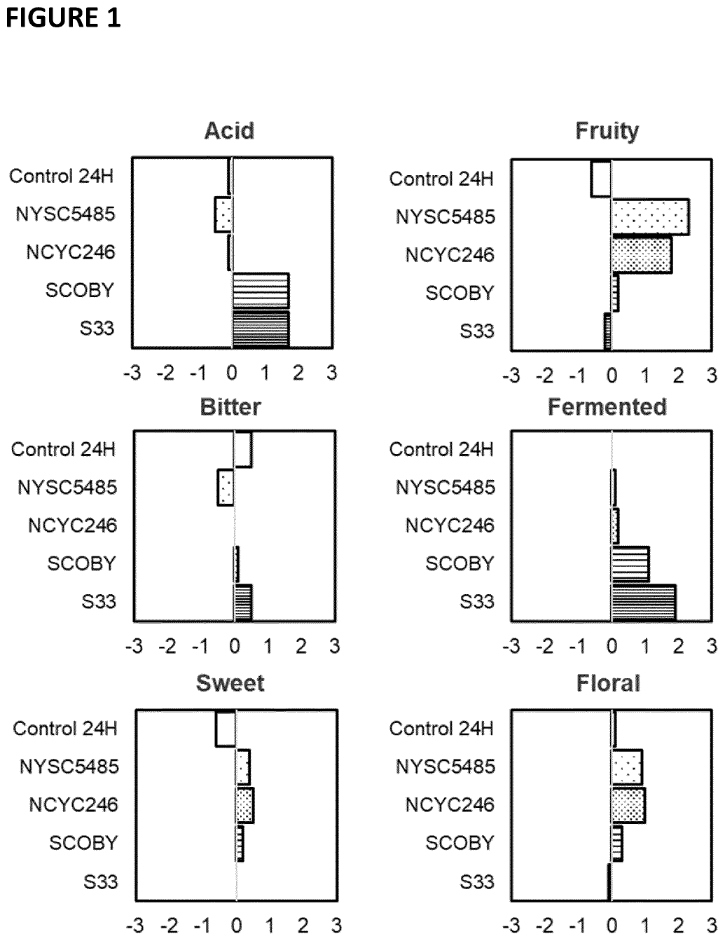

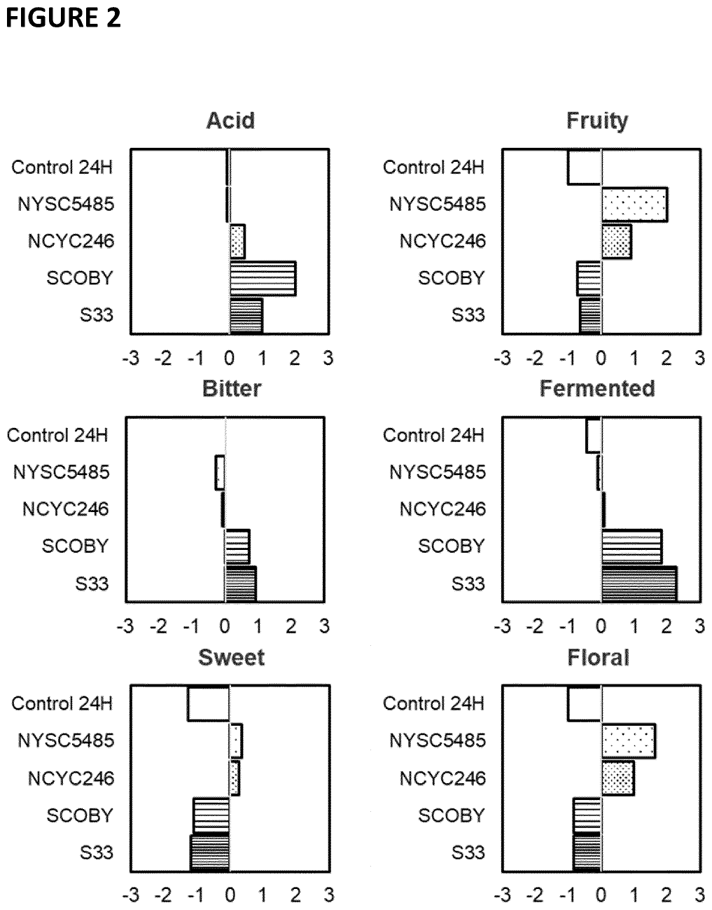

The results are plotted in FIG. 1 (cascara from Brazil) and FIG. 2 (cascara from Mexico). The samples fermented with a Pichia yeast (NYSC5485 and NCYC246) have enhanced sweet, fruity and floral notes. P. kluyveri NYSC 5485 provides greater fruity intensity than the type strain P. kluyveri NCYC 246.

### Example 3: Analysis of Aroma Compounds Present in Cascara from Mexico Fermented with Different Yeasts

Semi-quantitative volatile profiling of the cascara infusions were conducted by headspace/solid phase microextraction coupled with gas chromatogram and mass spectrometry (HS/SPME-GC-MS). The apparatus used was a gas chromatograph coupled with a single quadrupole mass spectrometer (Agilent Technology 5973N).

Initially, 1.0 ml of the cascara infusion samples were incubated in a 10-mL screw-top headspace vial at 30° C. for 10 min, followed by extraction using a SPME fiber (PDMS/DVB, Supelco) at 30° C. for 10 min. All vials were placed in a tray cooled at 6° C. before analysis. The volatiles were thermally desorbed from the SPME fiber at 250° C. in splitless mode and resolved with a s capillary column (DB-WAX, Agilent). The GC oven temperature was programed initially at 35° C. for 5 min, then raised to 230° C. at 4° C./min, then at 230° C. for 10 min. Helium was used as the carrier gas at a flow rate of 1 mL/min. The targeted compounds were identified by pure standards and quantified in MSD ChemStation software (Agilent). All samples were measured in duplicate. The concentration of ethanol was measured by headspace coupled with GC and flame ionization detection (HS-GC-FID, Agilent Technology 7890B). 1.0 ml of each sample was incubated in a 10-ml headspace vial for 5 min at 40° C., and then 1 ml was sampled and injected into the GC with a split ratio of 5. The GC oven temperature was programed initially at 35° C. for 1 min, then raised to 70° C. at 4° C./min, then to 180° C. at 12° C./min, and then held for 2 min. Helium was used as the carrier gas at a flow rate of 2 mL/min. All samples were measured in duplicate.

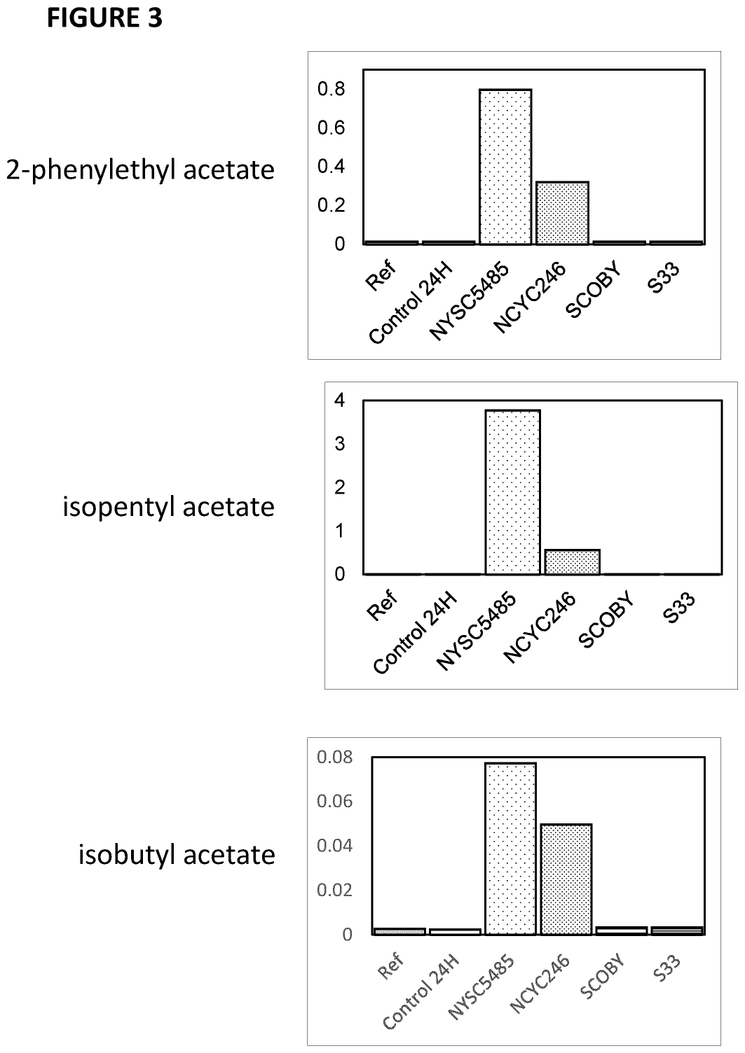

Levels of 2-phenylethyl acetate, isopentyl acetate and isobutyl acetate in parts per million are plotted in FIG. 3. The samples fermented with a Pichia yeast (NYSC5485 and NCYC246) have elevated levels of these acetate esters with P. kluyveri NYSC 5485 delivering the highest levels. Weight ratios of aroma components are given in the table below

| | | | | | S. |
|---|---|---|---|---|---|
| | | P. kluyveri | P. kluyveri | | cerevisiae |
| Ratio | Control 24H | NYSC 5485 | NCYC 246 | SCOBY | S-33 |
| 2-Phenylethyl | 0.5 | 28.3 | 17.5 | 0.6 | 0.6 |
| acetate/Benzaldehyde | | | | | |
| Isopentyl acetate/Benzaldehyde | 0.3 | 134 | 30.9 | 0.3 | 0.3 |
| Isobutyl acetate/Benzaldehyde | 0.1 | 2.7 | 2.7 | 0.1 | 0.1 |
| Isopentyl acetate/3-Methylbutanol | — | 2.7 | 3.2 | 0.2 | — |
| Isopentyl acetate/Hexyl acetate | 3.4 | 1262 | 155 | 3.6 | 3.9 |
| Ethyl acetate/Benzaldehyde | — | 990 | 1229 | 14.5 | 5.1 |
| Ethanol/Benzaldehyde | 428 | 27522 | 28069 | 18729 | 99052 |
| 2-Phenylethanol/Benzaldehyde | 1.2 | 18.4 | 12.0 | 2.6 | 151.6 |

The amount of ethanol produced after 24 hours fermentation with P. kluyveri NYSC 5485 was 0.03%.

The samples fermented with a Pichia yeast (NYSC5485 and NCYC246) show higher ratios of 2-phenylethyl acetate/benzakdehyde, isopentyl acetate/benzakdehyde, isobutyl acetate/benzaldehyde, isopentyl acetate/3-methylbutanol and isopentyl acetate/hexyl acetate than the control and the samples fermented with S. cerevisiae or SCOBY. P. kluyveri NYSC 5485 delivers higher values for these ratios than the type strain P. kluyveri NCYC 248.

### Example 4: Comparison of Genetic Sequence of P. Kluyveri NYSC 5485 to the Type Strain P. Kluyveri NCYC 246

The genetic sequence of Pichia kluyveri NYSC 5485 (CNCM 1-5525) and P. kluyveri NCYC 246 were analysed using PacBio sequencing technology. DNA purification was performed using a Qiagen Gentra Puregene Yeast kit. Twelve DNA fragments were identified for P. kluyveri NYSC 5485 and 15 for the type strain P. kluyveri NCYC 246. The genetic sequences for P. kluyveri NYSC 5485 are filed in electronic form as SEQ ID NO:1-SEQ ID NO:12 and the genetic sequences for P. kluyveri NCYC 246 filed as SEQ ID NO:13-SEQ ID NO:27. The mitochondrion DNA was SEQ ID NO:4 for P. kluyveri NYSC 5485 and SEQ ID NO:20 for P. kluyveri NCYC 246.

The genetic sequence of Pichia kluyveri NYSC 5485 (CNCM 1-5525) was compared with that of P. kluyveri NCYC 246 using the software OrthoANIu. The global average nucleotide identity was found to be 97.91%. Correspondences between the different sequences are given in the table below. Some sequences for Pichia kluyveri NYSC 5485 correspond most closely with a combination of sequences for P. kluyveri NCYC 246 and vice versa.

| Pichia kluyveri | P. kluyveri | Average nucleotide |
|---|---|---|
| NYSC 5485 | NCYC 246 | identity |
| SEQ ID NO: 7 | SEQ ID NO: 16 | 98.16% |
| SEQ ID NO: 10 | | |
| SEQ ID NO: 5 | SEQ ID NO: 22 | 97.90% |
| SEQ ID NO: 12 | | |
| SEQ ID NO: 11 | | |
| SEQ ID NO: 1 | SEQ ID NO: 13 | 97.87% |
| | SEQ ID NO: 15 | |
| SEQ ID NO: 3 | SEQ ID NO: 14 | 97.73% |
| SEQ ID NO: 8 | SEQ ID NO: 17 | |
| SEQ ID NO: 6 | SEQ ID NO: 27 | 97.94% |
| SEQ ID NO: 9 | | |
| SEQ ID NO: 2 | | |
| SEQ ID NO: 4 | SEQ ID NO: 20 | 99.02% |

The type strain P. kluyveri NCYC 246 comprised a genetic sequence with a maximum identity to each SEQ ID NO:1-12 taken individually as follows: SEQ ID NO:1, 97.85%; SEQ ID NO:2, 97.40%; SEQ ID NO:3, 97.86%; SEQ ID NO:4, 99.02%; SEQ ID NO:5, 98.05%; SEQ ID NO:6 97.93%; SEQ ID NO:7, 98.12%; SEQ ID NO:8 97.07%; SEQ ID NO:9, 97.20%; SEQ ID NO:10, 98.33%; SEQ ID NO:1, 197.526%; SEQ ID NO:12, 97.77%.

Various features and embodiments of the present invention will now be described with reference to the following numbered paragraphs (paras).

1. A beverage composition comprising fermented cascara or fermented cascara extract wherein the weight ratio of isopentyl acetate to benzaldehyde is greater than 1.

2. The beverage composition of para 1 wherein the weight ratio of isobutyl acetate to benzaldehyde is greater than 0.5.

3. The beverage composition of any one of paras 1 to 2 wherein the weight ratio of isopentyl acetate to 3-methylbutanol is greater than 0.5.

4. The beverage composition of any one of paras 1 to 3 wherein the weight ratio of isopentyl acetate to hexyl acetate is greater than 10.

5. The beverage composition of any one of paras 1 to 4 wherein the weight ratio of 2-phenylethyl acetate to benzaldehyde is greater than 0.9.

6. A beverage composition comprising fermented cascara or fermented cascara extract wherein the weight ratio of isobutyl acetate to benzaldehyde is greater than 0.5.

7. The beverage composition of para 6 wherein the weight ratio of isopentyl acetate to 3-methylbutanol is greater than 0.5.

8. The beverage composition of para 6 or para 7 wherein the weight ratio of isopentyl acetate to hexyl acetate is greater than 10.

9. The beverage composition of any one of paras 6 to 8 wherein the weight ratio of 2-phenylethyl acetate to benzaldehyde is greater than 0.9.

10. The beverage composition of any one of paras 6 to 9 wherein the weight ratio of isopentyl acetate to benzaldehyde is greater than 1.

11. A beverage composition comprising fermented cascara or fermented cascara extract wherein the weight ratio of isopentyl acetate to 3-methylbutanol is greater than 0.5.

12. The beverage composition of para 11 wherein the weight ratio of isopentyl acetate to hexyl acetate is greater than 10.

13. The beverage composition of para 11 or para 12 wherein the weight ratio of 2-phenylethyl acetate to benzaldehyde is greater than 0.9.

14. The beverage composition of any one of paras 11 to 13 wherein the weight ratio of isopentyl acetate to benzaldehyde is greater than 1.

15. The beverage composition of any one of paras 11 to 14 wherein the weight ratio of isobutyl acetate to benzaldehyde is greater than 0.5.

16. A beverage composition comprising fermented cascara or fermented cascara extract wherein the weight ratio of isopentyl acetate to hexyl acetate is greater than 10.

17. The beverage composition of para 16 wherein the weight ratio of 2-phenylethyl acetate to benzaldehyde is greater than 1.

18. The beverage composition of paras 16 or 17 wherein the weight ratio of isopentyl acetate to benzaldehyde is greater than 1.

19. The beverage composition of any one of paras 16 to 18 wherein the weight ratio of isobutyl acetate to benzaldehyde is greater than 0.5.

20. The beverage composition of any one of paras 16 to 19 wherein the weight ratio of isopentyl acetate to 3-methyl butanol is greater than 0.5.

21. A beverage composition comprising fermented cascara or fermented cascara extract wherein the weight ratio of 2-phenylethanol to benzaldehyde is greater than 2.7 (for example greater than 2.8, 2.9, 3.0) and the weight ratio of ethanol to benzaldehyde is less than 50000 (for example less than 40000, for further example less than 30000).

22. A method of preparing a beverage composition comprising;
adding a starter culture of yeast to coffee cherry pulp, wherein the starter culture provides at least 10⁴ CFU (for example at least 10⁵ CFU, for further example at least 10⁸ CFU) of yeast per gram of coffee cherry pulp; fermenting the coffee cherry pulp;
drying the fermented coffee cherry pulp to form fermented cascara and either;
extracting the fermented cascara with water to form a ready-to-drink beverage, or;
filling the fermented cascara into a container, the container being for the preparation of a beverage when inserted into a beverage preparation device.

23. The method of para 22 wherein the yeast is Pichia kluyveri, for example Pichia kluyveri NYSC 5485 (CNCM 1-5525).

24. The method of para 22 or para 23 wherein the beverage composition is according to any one of paras 1 to 21.

25. The method of any one of paras 22 to 24 wherein the beverage composition is according to any one of paras 32 to 37.

26. The method of any one of paras 22 to 25 wherein the coffee cherry pulp is essentially free from coffee beans.

27. The method of any one of paras 22 to 26 wherein the coffee cherry pulp is provided in the form of coffee cherries comprising coffee beans and the beans are removed after fermentation.

28. The method of any one of paras 22 to 27 wherein the fermentation occurs at a solids content greater than 40 wt. %, for example greater than 45 wt. %, for further example greater than 50 wt. %.

29. The method of any one of paras 22 to 28 wherein the fermented cascara is filled into a container such that the container contains fermented cascara and roast and ground coffee.

30. Use of coffee cherry pulp fermented with Pichia kluyveri NYSC 5485 (CNCM 1-5525) to produce a beverage.

31. A beverage composition comprising fermented cascara and/or fermented cascara extract wherein the weight ratio of 2-phenylethyl acetate to benzaldehyde in the fermented cascara and/or fermented cascara extract is greater than 0.7, for example greater than 0.8, 0.9, 1.0, 1.5, 3, 6, 10, 15, 20, 25, 30.40 or 50.

32. The beverage composition of para 31 wherein the weight ratio of isopentyl acetate to benzaldehyde in the fermented cascara and/or fermented cascara extract is greater than 1, for example greater than 1.5, 3, 6, 10, 15, 20, 25, 30, 40, 50, 60 or 70.

33. The beverage composition of para 31 or para 32 wherein the weight ratio of isobutyl acetate to benzaldehyde in the fermented cascara and/or fermented cascara extract is greater than 0.5, for example greater than 1, 2, 3 or 4.

34. The beverage composition of any one of para 31 to para 33 wherein the weight ratio of isopentyl acetate to 3-methylbutanol in the fermented cascara and/or fermented cascara extract is greater than 0.5, for example greater than 1, 2, 3 or 4.

35. The beverage composition of any one of para 31 to para 34 wherein the weight ratio of isopentyl acetate to hexyl acetate in the fermented cascara and/or fermented cascara extract is greater than 10, for example greater than 100 or 500.

36. The beverage composition of any one of para 31 to para 35 comprising roast and ground coffee or soluble coffee.

37. A container containing a composition comprising (for example consisting of) dried fermented coffee cherry pulp, the container being for the preparation of a beverage when inserted into a beverage preparation device.

38. The container of para 37 wherein the composition comprises roast and ground coffee.

1. A beverage composition comprising fermented cascara or fermented cascara extract wherein the weight ratio of 2-phenylethyl acetate to benzaldehyde is greater than 1.

2. The beverage composition of claim 1 wherein the weight ratio of isopentyl acetate to benzaldehyde is greater than 1.

3. The beverage composition of claim 1 wherein the weight ratio of isobutyl acetate to benzaldehyde is greater than 0.5.

4. The beverage composition of claim 1 wherein the weight ratio of isopentyl acetate to 3-methylbutanol is greater than 0.5.

5. The beverage composition of claim 1 wherein the weight ratio of isopentyl acetate to hexyl acetate is greater than 10.

6. The beverage composition of claim 1 wherein the beverage composition is selected from the group consisting of a ready to drink beverage, a beverage concentrate, a soluble beverage powder, dried aromatic plant material and combinations of these.

7. A container for use in a beverage preparation device, the container containing the beverage composition comprising fermented cascara or fermented cascara extract wherein the weight ratio of 2-phenylethyl acetate to benzaldehyde is greater than 1.

8. (canceled)

9. A method of preparing a beverage composition comprising;
a. steeping cascara in water to form an aqueous cascara extract and steeped cascara;
b. fermenting the aqueous cascara extract in the presence or absence of the steeped cascara; and
wherein the fermentation is performed by yeast in the absence of acetic acid bacteria.

10. The method of claim 9 wherein the yeast comprises Pichia kluyveri.

11. The method of claim 9 wherein the yeast comprises a genetic sequence having a global nucleotide identity of at least 98% to the combination of SEQ IDs NO:1-12.

12. The method of claim 9 wherein the fermentation is performed in the absence of Saccharomyces cerevisiae.

13. The method of claim 9 comprising separating the aqueous cascara extract from the steeped cascara after fermentation.

14. The method of claim 9 wherein the steeped cascara, present during the fermentation of the aqueous cascara extract, is dried together with the aqueous cascara extract after fermentation.

15. (canceled)
